(19) United States
(12) Patent Application Publication
(10) Pub. No.: US 2019/0233760 A1
(43) Pub. Date: Aug. 1, 2019

(54) METHOD FOR APPLYING A TREATMENT AGENT TO A SUBSTRATE

(71) Applicant: Xeros Limited, Rotherham, South Yorkshire (GB)

(72) Inventors: Aidan LAVERY, Rotherham, South Yorkshire (GB); Mehrin CHOWDHURY, Rotherham, South Yorkshire (GB)

(73) Assignee: Xeros Limited, Rotherham, South Yorkshire (GB)

(21) Appl. No.: 16/318,192

(22) PCT Filed: Aug. 15, 2017
(86) PCT No.: PCT/GB2017/052398

§ 371 Date: Jan. 16, 2019

(30) Foreign Application Priority Data
Aug. 15, 2016 (GB) 1613970.1

(51) Int. Cl. C11D 1/00; C11D 3/37; C11D 3/40; C11D 3/386; C11D 11/00; A47L 15/00; A47L 15/42; D06L 4/40; D06L 4/75; C11D 3/48

(52) U.S. Cl. CPC C11D 1/008; C11D 3/37; C11D 3/40; C11D 3/386; C11D 3/48; A47L 15/0002; A47L 15/4236; D06L 4/40; D06L 4/75; C11D 11/0017

(57) ABSTRACT

A method for applying a treatment agent to a substrate, wherein the treatment agent is bound to a solid polymeric particle at a first pH, and wherein the substrate is contacted with the solid polymeric treatment particles under conditions such that the treatment agent is released from the solid polymeric treatment particles.

The present invention relates to a method for applying a treatment agent to a substrate and to the applications of said method. The method is particularly useful for the application of treatment agents to textiles and fabrics, for example is laundry and textile processing, laundry and textile pre-treatments and dishwashing. The method can also be used for the application of dyes to substrates such as textiles and fabrics.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise. Thus, for example, a substrate means one or more substrates.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

Traditional wet cleaning of textiles and the like relies on the washing action provided by large quantities of water, in combination with appropriate detergent formulations. These detergents typically comprise a combination of surfactants, with or without various components selected from enzymes, oxidising or bleaching components optionally with associated activators, builders to control water hardness, anti-redeposition additives to prevent resettling of removed stain back on to the textile surface, perfumes, and optical brighteners to further mask the effects of redeposition, particularly on white garments.

WO-A-2007/128962 discloses a method for cleaning a soiled substrate, comprising the treatment of the moistened substrate with a formulation comprising a multiplicity of polymeric particles and requires the use of only limited amounts of water and detergents, thus offering a significant environmental benefit.

A problem with conventional wet cleaning processes, where the detergent formulation is usually added as an all-in-one dosing, is that there is a significant dilution of certain components of the detergent formulation at the textile surface as the wash progresses, Thus, good cleaning can occur at the expense of anti-redeposition additives, perfumes and optical brighteners being removed prematurely from the cleaned textile. These three parts of the detergent formulation are crucial in meeting consumer needs alongside cleaning quality. Hence, in conventional wet cleaning processes, all-in-one detergent formulations are effectively overloaded with these components, in order to ensure that they remain present in sufficient quantities on the final cleaned textile surface. This increases both the overall chemical loading in the wash, the cost of the detergent formulation itself, and the environmental cost of excess chemicals entering waste water systems.

One solution to these problems is proposed by WO-A-2011/128680, which describes a modified detergent dosing process for use when cleaning textiles using polymeric particles. In this method, the detergent formulation is split into its constituent chemical parts, with these being added at different times during the cleaning process, specifically during the wash and rinse sections of the cycle. In this way, not only is the overall chemical loading reduced, but the more expensive components of the formulations can be added when they are likely to be most effective, As a consequence, considerable cost savings are achieved when compared with conventional all-in-one detergents.

Another solution to these problems is proposed by WO-A-2014/0006424, which discloses a cleaning formulation where cleaning agents were immobilised on the surface of solid polymeric cleaning particles. Of course, the word “immobilisation” does not connote cleaning agents which can be released or applied to a substrate.

PCT patent publication WO2006/040539 discloses a method for the application of a substance to a substrate using a multiplicity of polymeric particles coated with at least one substance. This publication discloses nothing with regard to the way in which release of the substance is effected or promoted.

Despite the progress to date, there remains a need in the art for improved processes for delivering treatment agents to substrates, such as textiles, with improved efficacy and reduced waste.

In a first aspect, the present invention provides a method for applying a treatment agent to a substrate using solid polymeric treatment particles, said method comprising:
a) providing solid polymeric treatment particles obtainable by at least partially coating solid polymeric particles with the treatment agent in the presence of an aqueous liquid medium, the liquid medium having a first pH, wherein:
   (i) the surface of the solid polymeric particles has a net positive or net negative charge at the first pH; and
   (ii) the treatment agent has a net positive or net negative charge within its chemical structure at the first pH;
wherein the sign of the net charge on the surface of the solid polymeric particles at the first pH is opposite to the sign of the net charge of the treatment agent at the first pH; and
b) contacting the substrate with the solid polymeric treatment particles from step a), in an aqueous liquid medium under conditions such that the treatment agent is released from the solid polymeric treatment particles.

In a second aspect, the present invention provides a method for applying a treatment agent to a substrate, said method comprising providing solid polymeric treatment particles comprising solid polymeric particles and a treatment agent, wherein the treatment agent is ionically bound to the surface of the solid polymeric particles, and contacting said solid polymeric treatment particles with the substrate in an aqueous liquid medium at under conditions such that the treatment agent is released from the solid polymeric treatment particles.

In accordance with the second aspect of the invention, the solid polymeric treatment particles are obtainable by at least partially coating solid polymeric particles with the treatment agent in the presence of an aqueous liquid medium, the aqueous liquid medium having a first pH, wherein:
(i) the surface of the solid polymeric particles has a net positive or net negative charge at the first pH; and
(ii) the treatment agent has a net positive or net negative charge within its chemical structure at the first pH;
wherein the sign of the net charge on the surface of the solid polymeric particles at the first pH is opposite to the sign of the net charge of the treatment agent at the first pH.

Preferably, at least one of the solid polymeric particles and the treatment agent has an isoelectric point wherein the term “isoelectric point” refers to the pH at which the solid polymeric particles or the treatment agent respectively carry no net electrical charge in the statistical mean. More preferably, at least the solid polymeric particles have an isoelectric point such that the solid polymeric particles have a net positive charge at pH below the isoelectric point and a net negative charge at pH above the isoelectric point. The first pH may therefore be selected according to the charge on the treatment agent, such that there is electrostatic attraction between the solid polymeric particles and the treatment agent.

In one embodiment, the solid polymeric particles may have a first isoelectric point and the treatment agent may have a second isoelectric point. At pH between the first and second isoelectric points, the solid polymeric particles and the treatment agent carry opposite charges meaning that there is electrostatic attraction between the solid polymeric particles and the treatment agent.

The treatment agent may suitably be released from the solid polymeric treatment particles by contacting the solid polymeric treatment particles with the substrate in an aqueous liquid medium at a second pH at which the net charge on the surface of the solid polymeric particles or the net charge on the treatment agent has changed such that the signs of the net charge on the surface of the solid polymeric particles is the same as the sign of the net charge of the treatment agent or such that there is no net charge on the surface of the solid polymeric treatment particles or no net charge of the treatment agent. For example, the second pH may be at or above the higher of the two isoelectric points of the solid polymeric particles and the treatment agent. Alternatively, the second pH may be at or below the lower of the two isoelectric points of the solid polymeric particles and the treatment agent.

Alternatively, the treatment agent may suitably be released from the solid polymeric treatment particles by contacting the solid polymeric treatment particles with the substrate in an aqueous salt solution.

The solid polymeric treatment particles may suitably contacted with the substrate with agitation. Agitation may be useful so as to ensure that the treatment agent is applied to the entire surface of the substrate with an even distribution.

The present invention can help to overcome stability issues with some treatment agents in solution, such as fragrance, enzymes and bleach all of which can have stability problems in aqueous conditions. As a consequence, it may also be possible to reduce the amounts of stabilizers which might otherwise be required. This is especially useful in the case where the treatment agent is an enzyme. In addition, the present invention offers a method by which treatment agents can be more effectively delivered, released and applied to a substrate. A noteworthy advantage of the present invention is that positively charged reagents can be delivered to the substrate with less interaction with the anionic components which are often present in solution. The present invention also enables the treatment agent to be delivered to the substrate more efficiently, thus minimising waste. This offers valuable environmental and processing advantages.

The present invention furthermore improves on the process of WO-A-2014/0006424 and WO2006/040539 in that it provides a means by which treatment agents can be coated onto the surface of the polymeric particles and then actively released in a subsequent step. By utilising the treatment particles to apply the treatment agent directly to the substrate surface and by controlling the release from the particle it is possible to avoid or reduce the requirement for the separate addition of, for example, detergent ingredients which may be in the form of a powder, liquid, tablet, capsule etc. Therefore, by utilising the method of the present invention different treatment agents can be released at different stages of the treatment, e.g. wash. As an example, anionic treatment agents can be released at high pH (pH>7, more favourably pH>8 and especially in the range of pH 8 to pH11 while cationic ingredients can be released at a pH<5. The present invention also provides a means by which expensive treatment agents can be recovered at the end of the washing process.

The word “net” in relation to net negative or positive charge refers to the overall charge when considering the total numbers of anionic and cationic groups and the pH of the aqueous liquid medium. As an example a treatment agent having one carboxylic acid group and one amine group within its chemical structure would be net negatively charged in a liquid medium having a pH of 10 and net positively charged in a liquid medium having a pH of 2.

The method of the present invention has numerous applications. These include, but are not limited to, laundry and textile washing, treatments, pre-treatments and post-treatments of textiles, dishwashing, household cleaning, personal care applications, pharmaceutical topical applications, paper processing, food processing, catalysts, agrochemical application, insecticide application, water purification and waste water treatment, coatings and surface treatments and protein binding and release.

Preferably, the method of the present invention is used for laundry and textile washing, treatments, pre-treatments and post-treatments of textiles and fibres, and dishwashing.

The specific treatment agent applied by the method of the present invention can be selected with regard to the nature of the substrate.

The substrate can be a textile or a fibre. Preferably the textile or fibre substrate comprises a polyamide (especially nylon), leather, wool, silk, a polyalkylene (especially polypropylene and polyethylene), a polyurethane, a polyester, an acrylic, or a polysaccharide (especially cellulosics such as cotton, tencel, viscose, linen and particularly denim). The textile or fibre can also comprise a copolymer or a blend of the above.

Preferably, the substrate comprises or consists of a fibre.

When the method of the present invention is used for dishwashing, the substrate preferably comprises or consists of a hard surfaced material. Examples of hard surfaced materials include metals, ceramics, glass, plastics and wood.

Preferably, for dishwashing, the substrate comprises or consists of a metal (e.g. pans and cutlery), glass (e.g. wine glasses) or a ceramic (e.g. plates, cups, bowls).

When the substrate is a textile or fibre then the treatment agent may be selected from any of the treatment agents used in this area of technology.

When the method of the present invention is used in dishwashing the treatment agent may be selected from any of the treatment agents used in this area of technology.

Preferably, the treatment agent comprises at least one ionic group at the first pH. This ionic group may be either an anionic group or a cationic group, the treatment agent may also comprise both anionic and cationic groups.

When the treatment agent comprises at least one anionic group at the first pH, then the anionic group is preferably selected from sulfo acids, phospho acids, carboxylic acids, phenolics, thiophenols, heterocyclic hydroxyl and thiol compounds.

When the treatment agent comprises at least one cationic group at the first pH, then the cationic group is preferably selected from ammonium, quaternary ammonium, guanidinium, biguanide, dimethylammonium, monomethylammonium, triphenylamine, benzalkonium, benzylammonium, ester quaternary, Au³⁺/Au⁺, Ag⁺, Zn²⁺, Cu⁺/Cu²⁺, Mn₂(TMTACN)₂(μ-O)₃²⁺, the cationic form of the Busch catalyst, Jacobson's catalysts, the Collins catalyst and cationic polymers.

When the treatment agent comprises both cationic and anionic groups they may be present in equal numbers as in a zwitterionic surfactant or one or other may predominate as in proteins or enzymes.

When the treatment agent comprises a protein or enzyme then many of the ionic groups carried by the protein or enzyme may be hidden within its three dimensional structure. In this instance, the available ionic groups on the surface of the protein or enzyme and their cationic/anionic balance are influential in determining how these agents will behave in the method of the present invention.

Preferably, the solid polymeric particles are coated with a single treatment agent. However, it is not excluded that the solid polymeric particles can be treated with multiple treatment agents, especially with multiple treatment agents carrying similarly charged groups. It is also envisaged that several batches of solid polymeric particles can be coated with different treatment agents and combined prior to contacting the solid polymeric treatment particles to the substrate, wherein the treatment agents may be released simultaneously or at varying times.

Preferably, the treatment agent is selected from a surfactant, a buffer, a sequestrant, a builder, a dye, a singlet oxygen generator, a bleach compound, a bleach activator, a bleach catalyst, a dispersant, an optical brightener, an antioxidant, an enzyme, a fragrance, a cyclodextrin, an antistatic agent, a UV protector, an antimicrobial agent, a fabric conditioner, an insecticide, an insect-repellant, a flame retardant, a water-repellant, an oxide or a mixture thereof.

Optionally, the treatment agent may be selected from a zeolite, a clay, an acid, or a base, any of which can be used separately or in any combinations with the above preferred treatment agents.

Suitable oxides include zinc, titanium, aluminium and silicon oxides, especially ZnO, TiO₂, Al₂O₃, SiO₂.

When the treatment agent is a surfactant, it can optionally be selected from non-ionic, anionic, cationic, ampholytic and zwitterionic surfactants. Non-ionic surfactants are less preferred.

When the treatment agent is a builder, it can optionally be selected from the alkali metal, ammonium and alkanolammonium salts of polyphosphates, alkali metal silicates, alkaline earth and alkali metal carbonates, aluminosilicates, polycarboxylate compounds, ether hydroxypolycarboxylates, copolymers of maleic anhydride with ethylene or vinyl methyl ether, 1,3,5-trihydroxybenzene-2,4,6-trisulphonic acid, and carboxymethyl-oxysuccinic acid, the alkali metal, ammonium and substituted ammonium salts of polyacetic acids such as ethylenediamine tetraacetic acid and nitrilotriacetic acid, and polycarboxylates such as mellitic acid, succinic acid, oxydisuccinic acid, polymaleic acid, benzene 1 ,3,5-tricarboxylic acid, carboxymethyloxysuccinic acid and soluble salts thereof.

When the treatment agent is a fragrance, it can optionally comprise alcohols, ketones, aldehydes, esters, ethers and nitrile alkenes, and mixtures thereof. Commercially available compounds offering desirable substantivity to provide good fragrance include Galaxolide® (1,3,4,6,7,8-hexahydro-4,6, 6,7,8,8- hexamethylcyclopenta(g)-2-benzopyran), Lyral (3- and 4-(4-hydroxy-4-methyl-pentyl) cyclohexene-1 -carboxaldehyde and Ambroxan® ((3aR,5aS,9aS,9bR)-3a,6,6,9a-tetramethyl- 2,4,5,5a,7,8,9,9b-octahydro-1 H-benzo[e][1] benzofuran).

When the treatment agent is an optical brightening agent, it can optionally be selected from stilbene derivatives, benzoxazoles, benzimidazoles, 1,3-diphenyl-2-pyrazolines, coumarin, 1,3,5-triazin-2-yls and naphthalimides. Examples of these compounds include, but are not limited to, 4,4′-bis[[6-anilino-4(methylamino)-1,3,5-triazin-2-yl]amino]stilbene-2,2′-disulphonic acid, 4,4′-bis[[6-anilino-4-[(2-hydroxyethyl)methylamino]-1,3,5-triazin-2-yl]amino]stilbene-2,2′-disulphonic acid disodium salt, 4,4′-bis[[2-anilino-4-[bis(2-hydroxyethyl)amino]-1,3,5-triazin-6-yl]amino]stilbene-2,2′-disulphonic acid disodium salt, 4,4′-bis[(4,6-dianilino-1 ,3,5-triazin-2-yl)amino]stilbene-2,2′-disulphonic acid, disodium salt, 7- diethylamino-4-methylcoumarin, 4,4′-bis[(2-anilino-4-morpholino-1 ,3,5-triazin-6-yl)amino]-2,2′-stilbenedisulphonic acid, disodium salt, and 2,5-bis(benzoxazol-2-yl)thiophene.

When the treatment agents is a bleach, it can optionally be selected from borax decahydrate, peroxygen compounds, including hydrogen peroxide, inorganic peroxy salts, such as perborate, percarbonate, perphosphate, persilicate, and mono persulphate salts (e.g. sodium perborate tetrahydrate and sodium percarbonate), and organic peroxy acids such as peracetic acid, monoperoxyphthalic acid, diperoxydodecanedioic acid, N,N′-terephthaloyl-di(6-aminoperoxycaproic acid), N,N′-phthaloylaminoperoxycaproic acid and amidoperoxyacid.

When the treatment agent is a bleach activator, it can optionally be selected from carboxylic acid esters such as tetraacetylethylenediamine and sodium nonanoyloxybenzene sulfonate.

When the treatment agent is a bleach catalyst, it can optionally be selected from transition metal bleach catalysts, especially iron and manganese containing transition metal bleach catalysts. Complexes of manganese in oxidation state II, III, IV or IV; or iron in oxidation states II and III all of which preferably contain one or more macrocyclic ligand(s) with N, NR, PR, O and/or S donor functions are particularly preferred. Ligands that comprise nitrogen donor functions are preferably used. It is in this case particularly preferred to use bleach catalyst(s) which comprise as macrocyclic ligand 1,4,7-trimethyl-1,4,7-triazacyclononane (Me-TACN), 1,4,7-triazacyclononane (TACN), 1,5,9-trimethyl-1,5,9-triazacyclododecane (Me-TACD), 2-methyl-1,4,7-trimethyl-1,4,7-triazacyclononane (Me/Me-TACN) and/or 2-methyl-1,4,7-triazacyclononane (Me/TACN). Suitable manganese complexes are for example [Mn^{III}₂(μO)₁(μOAc)₂(TACN)₂](ClO₄)₂,[Mn^{III}Mn^{IV}(μO)₂(μ-OAc)₁(TACN)₂]-(BPh₄)₂, [Mn^{IV}₄(μ- O)₆(TACN)_{4]}(ClO₄)₄, [Mn^{III}2(μ-O)₁(μ-OAc)₂(Me-TACN)_{2]}(ClO₄)₂, [Mn^{III}Mn^{IV}(μ-O)₁(μ-OAc)₂(Me-TACN)₂](ClO₄)₃, [Mn^{IV}₂(μ-O)₃(Me-TACN)₂](PF₆)₂ and [MN^{IV}₂(μ-O)₃(Me/Me-TACN)₂](PF₆)₂(OAc═OC(O)CH₃). Said bleach catalysts are especially suitable for use with dishwashing or hard surfaced materials.

When the treatment agent is a dispersant, it can optionally be selected from homo- or co-polymeric polycarboxylic acids, or their salts, in which the polycarboxylic acid may comprise at least two carboxyl radicals separated from each other by not more than two carbon atoms.

In one preferred embodiment, the method of the present invention has particular utility when the treatment agent is an enzyme. Preferred enzymes include, but are not limited to, hemicellulases, proteases, cellulases (including those enzymes active at acidic and neutral/alkali pH), xylanases, lipases, phospholipases, esterases, cutinases, pectinases, keratinases, reductases, oxidases, phenoloxidases, lipoxygenases, ligninases, pullulanases, tannases, pentosanases, malanases, β-glucanases, arabinosidases, hyaluronidase, chondroitinase, laccase, mannanase and amylases, or mixtures thereof.

In another preferred embodiment, the method of the present invention has particular utility when the treatment agent is a dye. Any suitable water-soluble dye mentioned in the Color Index International may be used. Preferably, the dyes have either an anionic or cationic character. Preferred anionic dyes can be reactive dyes, direct dyes, acid dyes or azoic dyes. Mixtures of dyes may also be used.

The solid polymeric particles may comprise either foamed or unfoamed polymeric materials. Furthermore, the solid polymeric particles may comprise polymers which are either linear, branched or crosslinked.

The solid polymeric particles preferably comprise or consist of a polyalkene (such as polyethylene and polypropylene), a polyamide, a polyester or a polyurethane. Preferably, however, said solid polymeric particles comprise polyamide or polyester particles, most particularly particles of nylon, polyethylene terephthalate or polybutylene terephthalate.

Optionally, copolymers of the above polymeric materials may be included in said solid polymeric particles.

The solid polymeric particles may have an average mass of from about 1 mg to about 1000 mg, or from about 1 mg to about 700 mg, or from about 1 mg to about 500 mg, or from about 1 mg to about 300 mg, or from about 1 mg to about 150 mg, or from about 1 mg to about 70 mg, or from about 1 mg to about 50 mg, or from about 1 mg to about 35 mg, or from about 10 mg to about 30 mg, or from about 12 mg to about 25 mg, or from about 10 mg to about 800 mg, or from about 20 mg to about 700 mg, or from about 50 mg to about 700 mg, or from about 70 mg to about 600 mg, or from about 20 mg to about 600 mg.

The average volume of the solid polymeric particles may be in the range of from about 5 to about 500 mm³, from about 5 to about 275 mm³, from about 8 to about 140 mm³, or from about 10 to about 120 mm³, or at least 40 mm³, for instance from about 40 mm³ to about 500 mm³, or from about 40 mm³ to about 275 mm³.

In order of increasing preference, the solid polymeric particles have an average surface area of no more than 1500 mm², no more than 1400 mm², no more than 1200 mm², no more than 1000 mm², no more than 800 mm², no more than 750 mm², no more than 500 mm², no more than 300 mm², no more than 200 mm² and no more than 100 mm² per particle.

In order of increasing preference, the solid polymeric treatment particles have an average surface area of at least 1 mm², at least 5 mm² and at least 10 mm² per particle.

Preferably, the solid polymeric particles have an average surface area of from 10 mm² to 500 mm², more preferably from 10 mm² to 300 mm², still more preferably 10 mm² to 200 mm² and especially 10 mm² to 100 mm² per particle.

The solid polymeric particles preferably have an average particle size of at least 1 mm, more preferably at least 2 mm and especially at least 3 mm.

The solid polymeric particles preferably have an average particle size no more than 70 mm, more preferably no more than 50 mm, even more preferably no more than 40 mm, yet more preferably no more than 30 mm, still more preferably no more than 20 mm yet more preferably no more than 10 mm, and especially no more than 8mm.

Preferably, the solid polymeric particles have an average particle size of from 1 mm to 20 mm, more preferably from 1 mm to 10 mm.

Solid polymeric particles which offer an especially prolonged effectiveness over a number of treatment cycles are those with an average particle size of at least 5 mm, preferably from 5 mm to 10 mm.

The above-mentioned particle sizes provide especially good treatment performance whilst also permitting the treatment particles to be readily separable from the substrate at the end of the cleaning method. Separation is especially problematic wherein the substrate is a fibre or a textile.

References herein to average particle size, average mass, average volume or average surface area shall preferably be understood as number averages. The determination of the average particle size, mass, volume or surface area is preferably performed by measuring the values for at least 10, more preferably at least 100 cleaning particles and especially at least 1000 cleaning particles.

The particle size is preferably the largest linear dimension (length). For a sphere, this equates to the diameter. The size is preferably determined using Vernier callipers.

When the substrate is a textile fabric the combination of particle size, shape, hardness and density of the solid polymeric treatment particles is such that the mechanical interaction of the particle with the fabric is preferably optimised, it being sufficient to provide effective treatment but, at the same time, gentle enough to so as not to cause significantly more fabric damage when compared with conventional aqueous processes (performed in the absence of the treatment particles). It is, in particular, the uniformity of the mechanical action generated by the chosen particles across the entire fabric surface that is influential in this regard. Such uniform mechanical action is also advantageous to localised and controlled application of the treatment agents from the solid polymeric treatment particles across the entire substrate surface.

The preferred particle parameters set out above allow for easy separation of the particles from the substrate at the end of the treatment method. The particle size and shape suitably minimise entanglement with the substrate, and the combination of suitable particle density and high free volume (ullage) in the washing machine tumbling process together promote particle removal. This is especially relevant in the case of textile and fabric treatment processes.

It is particularly preferred that the solid polymeric particles comprise or consist of a polyamide. Preferably the polyamide comprises or consists of nylon 4,6; nylon 4,10; nylon 5; nylon 5,10;

nylon 6; nylon 6,6; nylon 6/6,6; nylon 6,6/6,10; nylon 6,10; nylon 6,12; nylon 7; nylon 9; nylon 10; nylon 10,10; nylon 11; nylon 12; nylon 12,12; silk and copolymers or blends thereof. More preferably the solid polymeric particles comprise or consist of nylon 6 or nylon 6,6; especially nylon 6.

The solid polymeric particles may carry either cationic (or cation-forming) or anionic (or anion-forming) groups. However, it is preferred that the solid polymeric particles carry both cationic (or cation-forming) and anionic (or anion-forming) groups.

Cationic and anionic groups may be incorporated into the solid polymeric particles via the inclusion of charged monomers in the polymerization or they may be introduced into the solid polymeric particles post polymerisation by a chemical reaction.

Preferably, the charged groups are present due to a discontinuity of polymer chains in the solid polymeric particles which results in unreacted end groups being exposed. In the case of nylon these unreacted end groups are amine and carboxylate groups which form cationic ammonium groups and anionic carboxylate groups, respectively at appropriate pH.

The solid polymeric particles preferably have an isoelectric point in the range of from pH 3 to pH 7 and more preferably in the range of from pH 4 to pH 6 and especially from pH 5 to pH 6. This is especially desirable when the solid polymeric particles carry both cation-forming and anion-forming groups.

When the solid polymeric particles comprise a mixture of polymer species, the isoelectric point is typically a range rather than a discrete value.

Isoelectric points may be conveniently measured by using equipment manufactured by Anton Paar, the Horiba IEP SZ 100 Autotitrator, the Malvern Multipurpose titrator MPT2 or by pH titration of anionic and cationic dyes onto the polyamide particle surface and monitoring the transmission spectra of the solutions to follow dye uptake. Preferably, the isoelectric point is determined using the Malvern Autotitrator MTP2 in a pH range of pH 2-12 at 20 ° C. in soft deioinised water. Preferably, the isoelectric point is measure in a liquid medium that comprises or consists of water, more preferably in soft water, especially in deionized water. The temperature of the liquid medium for the isoelectric point measurement is preferably 20° C.

When the solid polymeric particles carry both cation-forming and anion-forming groups, then at a low pH, which is below the isoelectric point, the solid polymeric particles will selectively bind anionic treatment agents and at a high pH, which is above the isoelectric point, the solid polymeric particles will selectively bind cationic treatment agents.

A particular advantage of solid polymeric particles which carry both cation-forming and anion-forming groups is that, after coating the solid polymeric treatment particles with ionic treatment agent, when the pH is changed there are two forces driving the treatment agent release from the solid polymeric treatment particles. Firstly, the charge on the group binding the treatment agent is lost, and secondly, charges with the same sign as that on the treatment agents develop on the solid polymeric particles and help repel the treatment agent from the solid polymeric particles.

The solid polymeric particles may also have a hydrophobic nature. Hydrophobic components may be incorporated into the solid polymeric particles via the inclusion of appropriate monomers in the polymerization or they may be introduced into the solid polymeric particles post polymerisation by a chemical reaction.

When the solid polymeric particles are in their isoelectric point range, and carry no overall charge, they may, especially if hydrophobic, selectively bind neutral and hydrophobic treatment agents.

Prior to use, the solid polymeric particles may be activated in order to improve their surface properties. Depending on the chemical nature of the polymer the activating step can utilise a chemical activating agent such as acids, bases, enzymes, oxidising agents or bleaches or a of physical activation treatment such as heat, electromagnetic radiation, energetic particles (such as electron beams) and plasma treatment.

When the solid polymeric particles comprises a polyamide then preferably the polyamide particles are activated with an acid, a base or an oxidising agent or a combination thereof prior to step a).

It is preferred that the polyamide particles are partially hydrolyzed and activated using a suitable acid. However, if the acid is too strong the polyamide will lose its structural integrity (for example the particles might swell or dissolve).

Thus, in a preferred treatment, polyamide particles are activated with an acid, prior to formation of the solid polymeric treatment particles, said acid preferably being hydrochloric acid, preferably having a molar strength of from 2.0 to 5 M, more preferably 2.0 to 4.0 M, even more preferably 3.0 to 4.0 M and most especially from 3.2 to 3.8 M.

The degree of hydrolysis will also depend on the time and temperature of the reaction. Thus, it has been found that an optimal activation of for polyamides (especially nylon 6,6) may be achieved by hydrolysis using 3.5 M hydrochloric acid, preferably at 50 ° C., preferably for 30 minutes optionally followed by washing in distilled water.

It is preferred that the solid polymeric treatment particles are obtained by a method as defined above in which the first pH is at least 1 pH unit above or 1 pH unit below the isoelectric point of the solid polymeric particles and more preferably at least 2 pH units above or 2 pH units below the isoelectric point of the solid polymeric particles.

It is particularly preferred that the isoelectric point of the solid polymeric particles is from 4 to 6 or from 4.5 to 6.5; and preferably the first pH is from 7 to 14, more preferably from 8 to 12 or from 1 to 5, more preferably 2 to 5; provided that the isoelectric point is not the same as the first pH.

Coating of the solid polymeric particles by the treatment agent is preferably carried out at a low temperature, preferably at a temperature of from 0° C. to 30° C., more preferably at a temperature of from 0° C. to 25° C. especially at a temperature 0° C. to 20° C. and more especially at a temperature of from 0° C. to 10° C.

Coating of the solid polymeric particles by the treatment agent is also preferably carried out in an aqueous liquid medium with a low ionic strength, preferably less than 0.1M and more preferably less than 0.01M.

Preferably, the coating of the solid polymeric particles by the treatment agent is carried out in a minimal amount of the aqueous liquid medium. Thus, the ratio of the aqueous medium to the solid polymeric particles on a weight basis should preferably be in the range of from 2:1 to 100:1, more preferably in the range of from 4:1 to 50:1 and especially in the range of from 5:1 to 20:1.

The optimum time required for coating the solid polymeric particles with the treatment agent will obviously depend on all the above factors plus the nature of the solid polymeric particles and the treatment agent. However, preferably, the coating step is performed for a duration of 1 minute to 24 hours and more preferably for a duration of 5 minutes to 3 hours.

Release of the treatment agent from the solid polymeric treatment particles is considered to be achieved by reducing the ionic attractive forces between the treatment agent and the surface of the solid polymeric particles. As set out above, this may be done by contacting the solid polymeric treatment particles with the substrate in an aqueous liquid medium at a second pH at which the net charge on the surface of the solid polymeric particles or the net charge on the treatment agent has changed such that the signs of the of the net charge on the surface of the solid polymeric particles is the same as the sign of the net charge of the treatment agent or such that there is no net charge on the surface of the solid polymeric treatment particles or no net charge of the treatment agent. For example, the second pH may be at or above the higher of the two isoelectric points of the solid polymeric particles and the treatment agent. Alternatively, the second pH may be at or below the lower of the two isoelectric points of the solid polymeric particles and the treatment agent.

Alternatively, the treatment agent may suitably be released from the solid polymeric treatment particles by contacting the solid polymeric treatment particles with the substrate in an aqueous salt solution (this is thought to vastly weaken any ionic attractive forces such that entropy and diffusion predominate).

The solid polymeric treatment particles may suitably be contacted with the substrate with agitation. Agitation may be useful to ensure that the treatment agent is applied to the entire surface of the substrate with an even distribution. When the release of the treatment agent from the solid polymeric treatment particles in step b) is achieved by the use of a salt solution, preferably to increase the ionic strength of the liquid medium, then the exact ionic strength preferred to release the treatment agent will be influenced by the nature of the treatment agent and the solid polymeric particles as well as other variables. However, in general, to release the treatment agent from the solid polymeric treatment particles the salt solution should preferably increase the ionic strength of the medium to in the range of from 0.1 M to 2 M.

Suitable salts for the salt solution include the alkali metal salts (especially Na and K) preferably in the form of the chloride, sulfate, nitrate or combination thereof.

The preferred method of release can be selected to best suit the application area, the nature of the treatment agent and the nature of the solid polymeric particles. It is preferred that release of the treatment agent from the solid polymeric treatment particles is achieved by changing the pH.

Preferably in the method of the present invention the first and second pH are selected such that:
A) the first pH is from 7 to 14; more preferably from 8 to 12; and the second pH is from 1 to 5, more preferably from 2 to 5; or
B) the first pH is from 1 to 5, more preferably from 2 to 5 and the second pH is from 7 to 14; more preferably from 8 to 12.

Optionally the release of the treatment agent from the solid polymeric treatment particles is achieved by changing the pH and increasing the ionic strength of the aqueous liquid medium.

There are other environmental factors which may help to promote the release of the treatment agent from the solid polymeric treatment particles. Thus, it is preferred that the solid polymeric treatment particles are contacted with the substrate at a temperature of from 20° C. to 90° C. and more preferably at a temperature of from 20° C. to 60° C.

The preferred time for release of the treatment agent from the solid polymeric treatment particles is influenced by all the above factors plus the nature of the solid polymeric particles and the treatment agent. However, preferably, this step is performed for a duration of from 1 minute to 3 hours and more preferably for a duration of from 5 minutes to 1.5 hours.

Preferably, the solid polymeric treatment particles are contacted with the substrate in the presence of a surfactant. The surfactant is preferably dissolved or dispersed in the aqueous liquid medium. The surfactant may be anionic, cationic or non-ionic. Preferably the surfactant is anionic, more preferably a sulphonate, especially an optionally substituted aryl sulphonate and most especially sodium dodecylbenzenesulphonate.

When the treatment agent is an enzyme, the surfactant is preferably a non-ionic surfactant more especially a polyethylene glycol containing surfactant. Preferred surfactants for enzymes comprise one or more —(CH₂CH₂O)— repeat units.

When the treatment agent is anionic then it is preferred that the surfactant is also anionic.

When the treatment agent is cationic then it is preferred that the surfactant is also cationic.

Preferably, the solid polymeric treatment particles are contacted with the substrate in a more dilute concentration than is used for the preparation of the solid polymeric treatment particles. Thus, the ratio of the aqueous liquid medium to the solid polymeric treatment particles is preferably in the range of from 2:1 to 100:1 more preferably in the range of from 4:1 to 50:1 and especially in the range of from 5:1 to 20:1.

Based on the above preferably:
i. the solid polymeric particles comprise or consist of a polyamide;
ii. the solid polymeric particles have an isoelectric point at a pH of from 4 to 6;
iii. the solid polymeric particles carry both cation-forming and anion-forming groups;
iv. the substrate comprises or consists of a fibre (more preferably the substrate comprises or consists of a textile);
v. the first and second pH are selected such that:
   A) the first pH is from 7 to 14; more preferably from 8 to 12; and the second pH is from 1 to 5, more preferably from 2 to 5; or
   B) the first pH is from 1 to 5, more preferably from 2 to 5 and the second pH is from 7 to 14; more preferably from 8 to 12.
vi. the treatment agent has one or more ionic groups;

The methods of the invention preferably further comprise steps c), d) and e):
c) separating the solid polymeric particles from the substrate and the aqueous liquid medium;
d) optionally cleaning the solid polymeric particles to remove any residual treatment agent;
e) re-using the solid polymeric particles in a method for applying a treatment agent to a fresh substrate as defined above.

Preferably the solid polymeric particles are reused one or more times in the method of the present invention, including the preferences as described above.

The solid polymeric treatment particles may be contacted with the substrate using an apparatus which comprises:
1. a housing;
2. a rotatable drum;
3. a motor configured so as to be capable of rotating the rotatable drum;
4. a pump for transporting the solid polymeric treatment particles into said drum; and
5. a sump for collecting the solid polymeric particles once the treatment is complete.

Such an apparatus is especially suited to a substrate which is or comprises a fibre or a textile. This apparatus is especially suited to use in laundry and textile washing, treatments, pre-treatments and post-treatments of textiles. This apparatus would often be described as a washing machine.

An apparatus such as this is disclosed in WO-A- 2010/094959, WO-A-2011/064581 and WO-A-201 1/098815.

The solid polymeric treatment particles may be contacted with the substrate using an apparatus which comprises:
1. a housing
2. a rack suitable for holding in place items to be washed;
3. one or more nozzles configured to be able to direct a stream of the aqueous liquid medium and solid polymeric treatment particles towards items when held on the rack;
4. a pump for pumping the solid polymeric treatment particles and the aqueous liquid medium through the one or more nozzles;
5. a sump for collecting the solid polymeric particles once the treatment is complete.

This apparatus is especially suited for use with a substrate which comprises or consists of a hard surfaced material as mentioned above. This apparatus is especially suited for use in dishwashing and will often be described as a dishwasher.

Solid polymeric treatment particles coated with a single treatment agent may be the only treatment particles used in the method of the present invention. Alternately, a mixture of solid polymeric treatment particles coated with different treatment agents may be used or a mixture of coated solid polymeric treatment particles and uncoated particles may be used in the method of the present invention.

The ratio of solid polymeric treatment particles to substrate can be in the range of from 30:1 to 0.1:1 w/w (dry mass of substrate (e.g. washload)), or in the region of from 10:1 to 1:1 w/w, or in a ratio of between 5:1 and 1:1 w/w, or around 2:1 w/w. These ratios have been found to be especially suitable for a substrate which comprises or consists of a fibre or textile. Thus, for example, 10 g of solid polymeric treatment particles would be employed for the cleaning of 5 g of fabric.

The methods of the present invention may be used for either small (≤10 Kg of dry substrate) or large scale (>10 Kg if dry substrate) batchwise processes, and find application in both domestic and industrial cleaning processes, The methods also find application in continuous processes, and in processes which combine batchwise and continuous operations.

The methods of the present invention can also be used to recover the treatment agent, particularly any unused treatment agent which has not been successfully been applied to the substrate. The recovery of the treatment agent can be partial or complete. This has two advantages, firstly the solid polymeric particles can be reloaded for use in a further treatment cycle and secondly it improves the environmental benefit of the above treatment methods by reducing the amounts of the residual treatment agent in the waste aqueous liquid medium.

For example, coating of the solid polymeric particles with the treatment agent may be carried out within the washing apparatus and subsequent release of the treatment agent would either be carried out in a separate washing cycle or outside the washing apparatus in a recovery step.

In accordance with the first aspect of the invention, steps a) and b) can be performed in the same apparatus or alternatively step a) and b) may be performed in different apparatus.

The methods of the invention may additionally comprise a step of fixing the treatment agent to the substrate and/or separating the solid polymeric treatment particles and the aqueous liquid medium from the substrate; and:
(i) adjusting the pH of the aqueous liquid medium to a third pH so as to provide a net positive or net negative charge on the surface of the solid polymeric particles, the third pH being such that the treatment agent has a net positive or net negative charge within its chemical structure, wherein the sign of the net charge on the surface of the solid polymeric particles is opposite to the sign of the net charge of the treatment agent; and/or
(ii) removing salts from the aqueous liquid medium.

In this way the attractive ionic forces are restored and any treatment agent which has not successfully applied to the substrate can be substantially or completely removed from the aqueous liquid medium.

As noted above, the method of the present invention has application in surface treatments. Preferred surfaces are the internal surfaces of an apparatus suitable for performing laundry and textile washing, treatments, pre-treatments and post-treatments of textiles and fibres, and dishwashing, and particularly apparatus in which solid polymeric particles or solid polymeric treatment particles (particularly solid polymeric treatment particles as defined herein) are used to treat a substrate within said apparatus. In use, the internal surfaces of such an apparatus may become soiled or unhygienic and require cleaning, such as by sterilization, disinfection or sanitization, for instance using an anti-microbial agent. The internal surfaces of such an apparatus include, for instance, the pipes, drums and filters within the apparatus. In the context of the present invention, therefore, the internal surfaces become the substrate to which a treatment agent is applied by said step of contacting with solid polymeric treatment particles.

Thus, in a preferred embodiment, the method of the present invention is a method of applying a treatment agent to a substrate wherein the substrate is selected from the internal surfaces of an apparatus suitable for performing laundry and textile washing, treatments, pre-treatments and post-treatments of textiles and fibres, and dishwashing. In this embodiment, preferred apparatus are those in which solid polymeric particles or solid polymeric treatment particles (particularly solid polymeric treatment particles as defined herein) are used to treat a first substrate within said apparatus, wherein the method of the present invention is used to apply a treatment agent to a second substrate wherein the second substrate is the internal surfaces of said apparatus. In this embodiment, the first substrate may be treated according to a method of the present invention, i.e. by applying a treatment agent to said first substrate as described hereinabove, wherein the treatment agent applied to the first substrate may be the same as or different to the treatment agent applied to the second substrate, and is preferably different. In this embodiment, said first substrate preferably comprises or consists of a fibre.

Thus, in a third aspect, the present invention provides a method of sterilizing, disinfecting or sanitizing an apparatus, said method comprising the steps of the first or second aspect of the present invention, wherein said substrate is selected from the internal surfaces of an apparatus. Particularly suitable apparatus are those noted hereinabove. Treatment agents for the third aspect are selected from any suitable sterilizing, disinfecting or sanitizing treatment agents conventional in the art, and are preferably selected from anti-microbial agents.

In a preferred embodiment of the third aspect of the invention, said internal surfaces of said apparatus are defined as a second substrate, and the method further comprises the additional steps of treating a first substrate with solid polymeric particles within said apparatus. The solid polymeric treatment particles are preferably solid polymeric treatment particles as defined herein and said additional steps are preferably the steps of the method of the first or second aspect of the present invention defined herein applied to said first substrate. In this embodiment, the treatment agent applied to said first substrate may be the same as or different to the treatment agent applied to said second substrate, and is preferably different. In this embodiment, said first substrate preferably comprises or consists of a fibre.

The preferred features of the first and second aspects of the invention described herein are also applicable to the third aspect of the invention.

The invention is further illustrated by the following Examples in which all parts and percentages are by weight unless otherwise stated.

### EXAMPLES

### Method of Hydrolysis, Adsorption & Desorption:

### Softened Water

All the water used in the examples below was softened using a standard water softener with an ion exchange column which was replenished daily using NaCl. This controls the Ca level to <5 ppm and the Mg level to <5 ppm.

### Equipment

A Konica Minolta Spectrophtometer CM-3600A with SpectraMagic NX Colour Data Software CM-S100w, Professional/Lite Ver2.2, as used to measure transmittance spectra and colour difference.

pH measurements were made using the VWRpH100L pH meter which was calibrated using the standard buffers also supplied by VWR with the pH meter at pH 4, pH 7 and pH 10.

### Hydrolysis of the Nylon 6 Particles

200 g of pure nylon particles (average diameter 4.3 mm, average surface area 58 mm²supplied by BASF, Ludwigshafen, Germany) were hydrolysed in a beaker using 200 mL, 3.5 M hydrochloric acid (diluted from 37% HCl, VWR Chemicals, UK) for 60 minutes at room temperature (20° C.) stirring the particles to ensure even surface exposure to the acid using a mechanical stirrer with a PTFE paddle. The hydrolysed nylon polymer particles were isolated from the HCI and buffered using acetic acid to pH 3.5 before being dried in a fume cupboard overnight at ambient temperature (20° C.). The dried particles were then rinsed with softened water before being used in the adsorption experiments as described in the following examples.

### Hydrolysis of Reactive Red 120 (RR120)

Hydrolysis of Reactive Red 120 was carried out by adding Reactive Red 120 (supplied by Sigma-Aldrich) (1 g, 7×10⁻⁴ M) to 80 mL of softened water with the pH adjusted to alkaline conditions by the addition of sodium carbonate until the pH was above pH 11 (at 80° C., pH 11.5) for 2 hours to ensure all of the dye's reactive component, mono-chlorotriazine, was converted to hydroxytriazine. The end of the reaction was apparent when the pH no longer reduced. The hydrolysed RR120 was then isolated as a solid.

### Example 1

### Adsorption of Hydrolysed Reactive Red 120

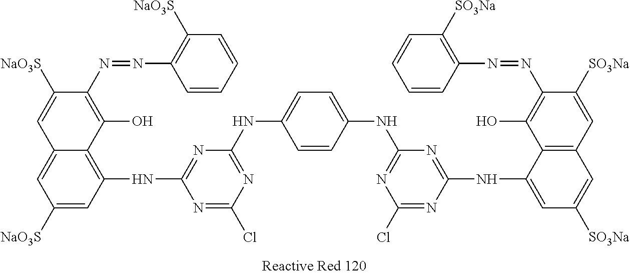

### Adsorption

Hydrolysed nylon 6 particles (50g) and unhydrolysed nylon particles (50g) were treated with 0.1 M HCl (VWR Chemicals, UK) at pH 2 for 30 minutes. Hydrolysed Reactive Red 120 (0.1 g, 7×10⁻⁵ M) was added, separately, to a solution containing softened water (30 mL) and 20 g of each of the nylon polymer particles (hydrolysed and unhydrolysed) and then left to adsorb the dye overnight in at 4° C. For each sample, the particles were separated and analysed using a CM-3600A spectrophotometer (Konica Minolta, UK) in a glass Quartz cuvette (Konica Minolta, UK; 1.3 cm width×3.8 cm length×5 cm height) at 540 nm.

### Desorption

The acid hydrolysed nylon 6 particles and unhydrolysed nylon particles on which hydrolysed RR120 dye had been absorbed were stirred in a beaker filled with 150 mL of soft water adjusted to pH 11 with sodium carbonate. 0.1 ml of Sodium dodecylbenzene sulphonate (SDBS; 30%, Univar Limited, UK) was added and the mixture was stirred at 60° C. for 30 minutes. For each sample, the particles were separated and analysed at 540 nm as described above.

### Results

Table 1 displays the results for the dye desorption from the polymer particle surface using Reactive Red. In Table 1 the lower the value of L* the more dye is adsorbed to the particle.

TABLE 1
L* data for adsorption
and desorption of hydrolysed Reactive Red 120

| Nylon 6 particle type | L* |
|---|---|
| Nylon 6 particles (no dye adsorbed) | 74.2 |
| Unhydrolysed particles after adsorption | 64.2 |
| of hydrolysed RR120 | |
| Unhydrolysed particles after desorption | 72.4 |
| of hydrolysed RR120 | |
| Hydrolysed particles with hydrolysed | 39.7 |
| RR120 adsorbed | |
| Hydrolysed particles after desorption | 51.8 |
| hydrolysed of RR120 | |

From these results it is clear that the anionic dye hydrolysed Reactive Red 120 is being bound at acidic pH and released at alkaline pH and the degree of binding of the dye is much greater when the hydrolysed nylon particles are used.

### Example 2

### Adsorption/Desorption of Methylene Blue (MB)

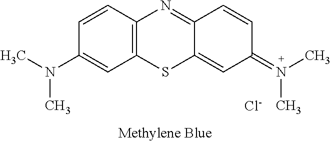

### Adsorption

Hydrolysed nylon 6 particles (50 g) and unhydrolysed nylon 6 particles (50 g) were treated with sodium hydroxide (VWR Chemicals, UK) at pH 12 for 30 minutes and then buffered with sodium carbonate to pH 12. Methylene Blue dye (0.04 g Sigma, UK) was then added to 800 mL of soft water at pH 7. In two separate experiments, the NaOH treated hydrolysed and unhydrolysed particles were left in the Methylene Blue solution at 4° C. overnight to adsorb the dye. For each sample, the particles were separated and then analysed using a CM-3600A spectrophotometer (Konica Minolta, UK) in a glass Quartz cuvette (Konica Minolta, UK; 1.3 cm width×3.8 cm length×5 cm height) at 670 nm.

### Desorption

The Methylene Blue dye was desorbed from the hydrolysed and unhydrolysed particles by washing with soft water at pH 3.5 and 40° C. in a beaker for 30 minutes. For each sample, the particles were separated and then analysed at 670 nm as described above. The results are shown in Table 2.

TABLE 2:
L* data for adsorption and desorption of Methylene Blue

| Nylon 6 particle type | L* |
|---|---|
| Pure nylon 6 particles | 74.2 |
| Unhydrolysed particles with MB adsorbed | 63.8 |
| Unhydrolysed particles after desorption of MB | 71.6 |
| Hydrolysed particles with MB adsorbed | 36.5 |
| Hydrolysed particles after MB desorption | 53.7 |

From these results, it is clear that the cationic dye Methylene Blue is being bound at alkali pH and released at acid pH and the degree of binding of the dye is much greater when the hydrolysed nylon particles are used.

### Example 3

### Washing Machine Experiments in the Beko WM5120W Automatic Washing Machine Front Loader

### Adsorption

Approximately 3 Kg of nylon 6 particles were hydrolysed with 11 M sodium hydroxide (NaOH, VWR Chemicals, UK) and then mixed with pH 11 sodium carbonate buffer (Sigma Aldrich, UK). Following this, in two separate experiments, 0.2 g of Methylene Blue dye was added to 4 L of soft water and then mixed with the unhydrolysed particles and NaOH hydrolysed particles before leaving them in the fridge overnight at 4° C. to allow dye adsorption. For each sample, the particles were separated and then analysed using a CM-3600A spectrophotometer (Konica Minolta, UK) in a glass Quartz cuvette (Konica Minolta, UK; 1.3 cm width×3.8 cm length×5 cm height) at 670 nm.

### Beko WM5102W Front Loading Washing Machine 5 kg Scale-Desorption

The washes were all carried out on the Cotton wash cycle at 40° C. Methylene Blue dye was desorbed from the 3 Kg of negatively charged particles by washing the particles using soft water (at pH 3.5) in a polyester bag and in the washing machine for 1 hour and 45 minutes in the presence of strips of cotton fabric (double scoured bleached cotton interlock fabric from Phoenix Calico Ltd, Huddersfield, UK). All desorption tests were repeated 3 times using the washing machine. Methylene blue pickup by the cotton fabric was analysed using a spectrophotometer, as detailed above, and L* was calculated (see Table 3). Control washes using the nylon 6 particles which had not been hydrolysed with NaOH and particle-free experiments were also undertaken. The results were as shown in Table 3.

TABLE 3
Dye Pick-up by Cotton fabrics from the hydrolysed, unhydrolysed
and No particle experiments with Methylene Blue Dye

| L* | | | |
|---|---|---|---|
| | Hydrolysed | Unhydrolysed | |
| Cotton fabric | particles | particles | No particles |
| Double scoured | 97.3 | 97.3 | 97.4 |
| bleached cotton | | | |
| interlock fabric | | | |
| wash 1 | 84.7 | 91.2 | 91.3 |
| wash 2 | 88.2 | 93.8 | 92.8 |
| wash 3 | 90.3 | 94.9 | 93.6 |

Here a lower value of L* indicates a higher level of dye uptake by the cotton fabric corresponding to a higher release of Methylene Blue from the nylon particles.

### Example 4

### Effect of pH on the Binding of the Anionic Dye Hydrolysed Reactive Red 120

Low pH solutions (i.e. pH 2, pH 4, pH 6, pH 7) were prepared using soft water (water hardness <5 mg/L) and acetic acid (VWR Chemicals, UK) and high pH solutions (i.e. pH 8, pH 10, pH 12) were prepared using sodium carbonate. The pH of the solutions was confirmed using a pH meter (VWR pH enomenal, pH 1100 L, UK). The pH meter was calibrated before sample measurements were taken. Hydrolysed Reactive Red 120 (0.0005 g) was added to 15 mL of the different pH solutions to obtain a molarity of 0.02 mM. Following this, 1.5 g of hydrolysed (using 3.5 M HCl) nylon particles were added to each pH solution and left in the fridge overnight at 4° C. to allow dye adsorption. For each sample, the particles were separated and the solution was then analysed using a CM-3600A spectrophotometer (Konica Minolta, UK) in a glass Quartz cuvette (Konica Minolta, UK; 1.3 cm width×3.8 cm length×5 cm height) at 540 nm. The transmittance measured is shown in Table 4, wherein the higher the transmittance, the more dye had bound to the particles and so been removed from solution.

Thus, clearly the acid hydrolysed nylon 6 particles are selectively binding the negatively charged hydrolysed Reactive Red 120 at low pH.

TABLE 4
Transmittance against different pH's for
Hydrolysed Reactive Red 120

| pH range | Transmittance/A.U @540 nm |
|---|---|
| pH 2 | 4 |
| pH 4 | 3.4 |
| pH 5 | 3.4 |
| pH 6 | 3.4 |
| pH 8 | 3.4 |
| pH 10 | 3.4 |
| pH 12 | 1.1 |

### Example 5

### Effect of pH on the Binding of the Cationic Dye Methylene Blue

Low pH solutions (i.e. pH 2, pH 4, pH 6, pH 7) were prepared using softened water and acetic acid and high pH solutions (i.e. pH8, pH10, pH12) were prepared using sodium carbonate solution to adjust the pH. The pH was confirmed using the VWR pH meter. Methylene Blue dye (0.0012 g) obtained from Sigma-Aldrich, was added to 15 ml of the different pH solutions to obtain a molarity of 0.0002 mM following which, 1.5 g of hydrolysed (using 11M NaOH) nylon 6 particles were added to each pH solution and left in the fridge overnight at 4° C. to allow dye adsorption. For each sample, the particles were separated and the solution was analysed at 670 nm using a CM-3600A spectrophotometer. The transmittance measured is shown in Table 5, wherein the higher the transmittance the more dye had bound to the particles and so been removed from solution. Thus, clearly the NaOH hydrolysed nylon 6 particles are selectively binding the positively charged Methylene Blue at high pH.

TABLE 5
Transmittance against different pHs
for Methylene Blue:

| pH range | Transmittance/A.U @670 nm |
|---|---|
| pH 2 | 3.5 |
| pH 4 | 3.7 |
| pH 5 | 3.7 |
| pH 6 | 3.7 |
| pH 8 | 4 |

Clearly the results in Tables 4 and 5 show that the hydrolysed nylon 6 particles are able to selectively bind cationic and anionic molecules at different pH values.

### Example 6

### Amylase Adsorption and Desorption

Hydrolysed nylon 6 particles (500 g) were buffered at pH 4.5 with citric acid for 60 minutes. The particles were then rinsed with pH4.5 buffer citric acid to ensure the pH was constant. Amylase (Stainzyme Plus 12L) from Novozymes, (12 mL of a 1/100 dilution) was added to 15 g of the hydrolysed nylon 6 particles and separately to 15 g of unhydrolysed nylon 6 particles. As a control, a solution of amylase without any particles was also provided. To ensure the enzyme was adsorbed onto the surface of the nylon polymer particles the samples above were all put on a roller and left there for 1 hour to provide good interaction between the enzyme in solution and the polymer particle surface. The particles and enzyme samples were then placed in the fridge at 4° C. for a further hour.

Protein Assay to Confirm Enzyme Adsorption onto Particles

Enzyme adsorption onto the nylon 6 polymer particles was determined by measuring the depletion of protein from solution onto the particle surface, using the BioRad DC assay version of the Bradford assay, ref: Bradford, M., Anal. Biochem., 72, 248 (1976), for protein concentration estimation. Each of the three test solutions (i.e. hydrolysed particles, unhydrolysed particles and the enzyme (without particles)) had 0.3 mL of particle free liquid removed. 1.5 mL of BioRad Reagent A and 12 mL of BioRad reagent B were then added. The three solutions were gently stirred and left to stand for 15 minutes and the blue colour which developed was monitored by its transmittance at 740 nm with a Konica Minolta CM-3600A spectrophotometer. The protein concentrations were estimated based on a standard curve of known protein (bovine serum albumin) concentration.

### Adsorption of Amylase

In the experiment, the enzyme solution, minus particles, is the control and there is no loss of protein. For the solution containing the hydrolysed nylon 6 particles, 94.4% of the protein was depleted from solution at pH 4.5 while in the solution containing the unhydrolysed particles 19% of the protein was depleted from solution at pH 4.5 (see Table 6 below). The difference between the two particles is attributed to the increased positive charge present on the surface of the hydrolysed nylon particles, which drives the enzyme adsorption through an electrostatic interaction.

TABLE 6
% Transmittance, protein concentration (mg/mL) and % adsorption values
for Amlyase enzyme only, hydrolysed particles and unhydrolysed particles

| | % Transmittance | Protein conc | |
|---|---|---|---|
| | @ 740 nm | (mg/mL) | % adsorption |
| Amylase enzyme | 82.88 | 0.140 | — |
| Hydrolysed particles | 89.94 | 0.008 | 94.4 |
| Unhydrolysed Particles | 83.82 | 0.113 | 19.0 |

### Desorption of Amylase

The hydrolysed nylon 6 particles from above were removed from their amylase solutions, dried and then stirred in a beaker filled with 100 mL of soft water at pH 10. Standard Industry/Commercial Laundry Monitor WFK 10R starch stained swatches were added to each of the solutions and stirred at 50° C. for 40 minutes. The same test was applied to a beaker containing an identical concentration of enzyme to that initially added to the polymer particle solutions as a control. The swatches were then left to dry at room temperature before spectrophotometric (Konica Minolta detailed previously) measurements were obtained. The L* data for each sample (hydrolysed particles and enzyme only) were then calculated and the details of the results were as tabulated below in Table 7.

TABLE 7
Washed stained swatch results for amylase enzyme only
and hydrolysed particles.

| | L* |
|---|---|
| Amylase enzyme | 77.9 |
| Hydrolysed particles | 78.4 |

The higher the L* value the more starch stain is removed from the cotton.

The data show that the particles released amylase from the polymer particle surface and this was effective and in turn helped in removing the starch stain from the cotton giving an improved performance over the amylase solution.

Clearly from the above experiment it is apparent that amylase may be bound to the nylon 6 particles at low pH and the released in an active form when the pH is raised.

### Example 7

### Absorption/Desorption of Surfactant

### 1) Adsorption

15 grams of pure Nylon 6 beads, unhydrolyzed, were buffered to pH=3 using dilute citric acid solution and then rinsed with soft water, dried and placed into a 12 mL solution of pH=10, 1 g/L sodium dodecyl benzene sulphonate (SDBS; Sigma Aldrich, UK), in 0.5M sodium carbonate (Sigma Aldrich, UK) at 21° C. for 1 hour with gentle agitation the solution was buffered to low pH=4, then left at 4° C. in the fridge for 16 hours overnight. The beads were then extracted from the sample solutions, rinsed with softened water (pCa<5 ppm) and dried. The same procedure was repeated for 15 g of hydrolysed Nylon beads (hydrolysed as described above).

### 2) Desorption

The dried Nylon polymer particles were placed in pH10 solution (Na₂CO₃, softened water) with 5 cm×5 cm stain swatches (EMPA) containing particulate soil. Desorption occurred by increasing pH to 10 and by increasing the temperature to 40° C. and the dilution in water to 100 ml. In these conditions, SDBS is released into solution and is adsorbed onto the cotton substrate.

### 3) Application to Particulate Soil Removal

Carbon black soot-stained swatches (supplied by WFK stain set with the soot stain 9ORM stain sets supplied by WFK Bruggen, Germany) were washed in 100 mL pH 10 solution water using 15 g of SDBS-treated hydrolysed beads or SDBS-treated unhydrolysed beads at 40° C. for 10 minutes in 500 mL glass beakers, and the amount of particulate soil removed was assessed using the Konica Minolta spectrophotometer. A control sample at pH7 (softened water), using neither beads nor SDBS was also tested. The hydrolysed beads demonstrate superior wash performance compared with the unhydrolysed beads (due to more SDBS being adsorbed onto the hydrolysed beads) and this stain removal continues for at least 5 washes, as illustrated in Table 8.

TABLE 8

| | ΔL*(vs the unwashed | |
|---|---|---|
| | black soot-stained swatch) | |
| | Wash 1 | Wash 5 |
| Control | 0.0 | 0.0 |
| Unhydrolysed Nylon 6 beads | 1.0 | 0.5 |
| Hydrolysed Nylon 6 beads | 5.4 | 5.5 |

### Example 8

### Absorption/Desorption of Optical Brightening Agent

### 1) Adsorption

15 g of pure Nylon 6 beads (unhydrolysed) were buffered to pH=3 using dilute citric acid solution (0.5M), rinsed with soft water, dried and placed into a 12.5 mL solution of pH=10, 10 g/L Leucophor BMB 2000 optical brightening agent (Clariant, Germany), in 0.5M sodium carbonate (Sigma Aldrich, UK) at 21° C. for 1 hour with gentle agitation and solution buffered to pH=4, then placed in the fridge at 4° C. for 16 hours overnight. The beads were then extracted from the sample solutions, rinsed with softened water (pCa<5 ppm) and dried. The same procedure was repeated for 15 g of hydrolysed Nylon 6 beads.

Uptake of OBA (optical brightening agent) to hydrolysed and unhydrolysed beads was analysed by UV-VIS analysis (Konica Minolta spectrophotometer) for the colour change of the beads after the adsorption of OBA. The date in Table 9 show that the uptake of OBA is superior for the hydrolysed beads.

TABLE 9

| | ΔL* |
|---|---|
| Unhydrolysed Nylon 6 beads | 4.8 |
| Hydrolysed Nylon 6 beads | 6.0 |

### 2) Desorption

The dried Nylon 6 polymer particles were placed in pH=10 solution (0.5M Na₂CO₃, softened water) with 5 cm×5 cm cotton swatches. Desorption occurred by increasing pH to 10 and by increasing the temperature to 40° C. and the dilution in water to 100 ml. In these conditions the OBA is released into solution and is adsorbed onto the cotton substrate (as measured by Konica Minolta spectrophotometer)

### 3) Application of OBA to Cotton Substrate

The amount of OBA absorbed onto the cotton swatches (cotton supplied by Phoenix Calico Ltd, Stalybridge, Manchester, Cheshire) after being released from the bead surface was evaluated as the difference, ΔL*, between the untreated cotton substrate and the treated cotton substrate. The results presented in Table 10 show that the hydrolysed beads release more OBA beads, and demonstrate superior performance, compared with the unhydrolyzed beads. This effect continues for at least 8 washes.

TABLE 10

| | ΔL* (vs the untreated cotton substrate) | |
|---|---|---|
| | Wash 1 | Wash 8 |
| Treated unhydrolysed beads | 0.8 | 1.2 |
| Treated hydrolysed beads | 2.3 | 1.7 |

### Example 9

### Absorption/Desorption of Fragrance

### 1) Adsorption

15 grams of pure Nylon 6 beads were buffered to pH=3 using dilute citric acid solution and then rinsed with soft water, dried and placed into a 12 mL solution of pH=10, 10 g/L o-Vanillin (Sigma Aldrich, UK), in 0.25 g/L sodium carbonate (Sigma Aldrich, UK) at 21° C. for 1 hour with gentle agitation buffered to pH=4, then at 4° C. in the fridge for 16 hours. The beads were then extracted from the sample solutions, rinsed with softened water (pCa<5 ppm) and dried. The same procedure was repeated for 15 g of hydrolysed Nylon beads.

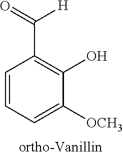 [SMILES: COc1cccc(C=O)c1O]

Uptake of o-Vanillin (λmax=450 nm) to the beads was analysed by UV-Vis analysis (Konica Minolta spectrophotometer) of the colour change of the beads on adsorption of coloured fragrance. The results in table 11 show that the hydrolysed beads adsorb more fragrance than unhydrolysed beads.

TABLE 11

| | ΔL* |
|---|---|
| Treated unhydrolysed beads | 12.8 |
| Treated hydrolysed beads | 16.1 |

The fragrance uptake by the polymer beads was also assessed by an experienced panel of fragrance assessors, and the results from that assessment (see Table 12) also demonstrated that an enhanced uptake of fragrance was observed for the hydrolysed beads.

TABLE 12

| | Panel for fragrance assessment | | | | |
|---|---|---|---|---|---|
| | of o-Vanillin on the polymer beads | | | | |
| | No smell | very weak | weak | strong | very strong |
| | Scale 1-5 | | | | |
| | 1 | 2 | 3 | 4 | 5 |
| Unhydrolysed beads | 3 | 6 | 4 | 0 | 0 |
| Hydrolysed beads | 2 | 0 | 2 | 6 | 3 |

### 2) Desorption

Desorption occurred by increasing pH to 10, increasing the temperature to 40° C. and dilution in soft water to 100 ml. In these conditions, o-Vanillin is released into solution and can subsequently be adsorbed onto the cotton substrate.

### 3) Application of Desorbed Fragrance to Cotton

The dried Nylon particles were placed in pH=10 solution (0.5M Na₂CO₃, softened water) with 5 cm×5 cm cotton swatches. The swatches were stirred in a 500 ml beaker with the vanillin beads and the fragrance was desorbed from the bead surface and onto the cotton swatches. An odour perception test was then carried out by a panel of 13 experienced textile fragrance assessors (see table 13) to assess the cotton swatches treated with o-Vanillin which had been desorbed from the hydrolysed Nylon beads and the unhydrolysed Nylon beads. The hydrolysed beads showed the greatest transfer of fragrance to the cotton and were superior in performance to both the unhydrolyzed beads and some free fragrance (control below) added in solution.

TABLE 13

| | Panel for cotton swatches with Vanillin | | | | |
|---|---|---|---|---|---|
| | No smell | very weak | weak | strong | very strong |
| | Scale 1-5 | | | | |
| | 1 | 2 | 3 | 4 | 5 |
| Unhydrolysed beads | 10 | 3 | 0 | 0 | 0 |
| Hydrolysed beads | 1 | 0 | 4 | 8 | 0 |
| Control fragrance only | 2 | 6 | 5 | 0 | 0 |

### Example 10

### Absorption/Desorption of Salicylic Acid

### 1) Adsorption

15 grams of pure hydrolysed Nylon 6 beads were buffered to pH=3 using 0.5M citric acid solution and then rinsed with soft water, dried and placed into a 12 mL solution at pH=10. 0.5% salicylic acid solution (Sigma Aldrich, UK), in 0.5M sodium carbonate (Sigma Aldrich, UK) was then added to the hydrolysed beads and the adsorption process carried out at pH=4, at 21° C. for 1 hour with gentle agitation, then these beads were placed in the fridge at 4° C. for 16 hours. The beads were then extracted from the sample solutions, rinsed with softened water (pCa<5 ppm) and dried. The same procedure was repeated for 15 g of unhydrolysed pure Nylon beads.

0.5 mL of the soak solutions of hydrolysed beads, non-hydrolysed beads were added to 10 mL of 1% ferric (III) chloride (Merck, Germany). A control sample which contained no beads was also prepared. The resulting change of colour for each sample was then compared to a calibration of known dilutions of salicylic acid and ferric (III) chloride to determine the concentration of the salicylic acid in the solutions for each sample calculated from the uptake of salicylic acid by each type of bead. The intensity of the colour indicates the concentration of salicylic acid. The control sample showed dark violet-blue colour indicating the presence of high concentration of the salicylic acid. The hydrolysed beads solution showed a light violet/brown colour because the salicylic acid had been adsorbed onto the positively charged polymer bead surface. The unhydrolysed beads sample displayed a fairly dark violet-blue colour approaching that of the control sample, indicating the presence of relatively high concentration of salicylic acid, but the colour differential indicated that a small amount of salicylic acid had been adsorbed onto the polymer bead surface.

### 2) Desorption

The dried polymer beads with the adsorbed salicylic acid were placed in a 500 ml beaker and 100 ml soft water added. The pH was increased from 4 to 10 using 0.5M sodium carbonate solution. In addition, the temperature was increased to 40° C. to drive the desorption of salicylic acid from the bead surface.

### 3) Application to Cotton

The beads were placed and stirred in a 500 mL beaker containing 100 mL soft water and a cotton swatch (1.5 g; 5 cm×5 cm). A control with salicylic acid but no beads was evaluated at the same time. The swatches were then dried and stirred with ferric (II) chloride solution for 5 minutes before rinsing with soft water and drying. The swatches were then analysed using the UV-VIS Spectrophotometer (Konica Minolta). The data in Table 14 demonstrate that the salicylic acid was transferred from the bead surface to the cotton with a slightly higher level of transfer for the hydrolysed beads.

TABLE 14

| Sample | ΔL* |
|---|---|
| Salicylic Acid | 13.1 |
| Unhydrolysed beads treated with salicylic | 16.2 |
| acid | |
| Hydrolysed beads treated with salicylic acid | 16.7 |

### Example 11

### Hygiene

### 1) Adsorption

Hydrolysed and unhydrolysed nylon beads were treated with Caflon BIT 20 (1 gram in 100 mL pH 10 solution (Univar, UK). Incubated over night at 4° C. Beads were then rinsed with soft water and dried. Then 500 uL of sump water (pH 10) was inoculated on the surface of the petri dish (nutrient+Blue+TTC; VWR Chemicals, Belgium). Pure nylon, hydrolysed and non-hydrolysed beads treated with Caflon BIT 20 were placed on the petri dish, and incubated at room temperature for 48 hrs before microscopic analysis was undertaken.

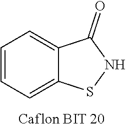

### 2) Application of Caflon BIT 20

Hydrolysed beads treated with Caflon BIT 20 displayed an inhibition zone and no bacterial growth. The unhydrolysed beads treated with Caflon BIT 20 showed some bacterial growth, but not as much growth as the untreated beads.
The average inhibition zone diameter for hydrolysed beads treated with Caflon BIT 20 is 2 cm×2 cm. On a scale of 1-4 (where, 1—no change, 2 small zone, 3—medium zone, 4—large zone), the average inhibition zone diameter was 4.
The average inhibition zone diameter for unhydrolysed beads treated with Caflon BIT 20 is 0.3 cm×0.3 cm. On the above-noted scale of 1-4, the average inhibition zone diameter was 2.

The results indicate that the hydrolysed beads with Caflon BIT 20 had excellent antimicrobial activity.

### Example 12

### Bleaching

### 1) Adsorption

15 grams of pure Nylon 6 beads were buffered to pH3 using dilute citric acid solution and then rinsed with soft water, dried and placed into a 12 mL solution of pH10, 10 g/L sodium perborate tetrahydrate (Sigma Aldrich, UK), in 0.25 g/L sodium carbonate (Sigma Aldrich, UK) at 21° C. for 1 hour with gentle agitation, then at 4° C. in the fridge for 16 hours. The beads were then extracted from the sample solutions, rinsed with softened water (pCa<5 ppm) and dried. The same procedure was repeated for 15 g of hydrolysed Nylon 6 beads.

### 2) Desorption

The dried Nylon polymer particles were placed in a 100 mL pH=10 solution with 5 cm×5 cm tea-stain swatches. Desorption occurred by (i) increasing pH from 4 to 10 (0.5M sodium carbonate), (ii) increasing the temperature to 40° C., and (iii) dilution in water to 100 mL. In these conditions, the hydroperoxide anion is released into solution and this is then subsequently adsorbed onto the curry stained cotton swatches.

### 3) Application to Curry Stain Removal

The curry stain was prepared using the following procedure. Cotton swatches (circular, with a diameter of 5 cm) were cut out using a template board. A small sponge was then used to apply the curry sauce (Morrison's own label curry sauce, Morrisons, UK) to ensure full coverage of the cotton swatches. The swatches were then left to dry at room temperature for 4 days before use. The curry-stained swatches were washed in 100 ml soft water at pH=10 using 15 g of sodium perborate-treated beads (both the hydrolysed and unhydrolysed beads) at 40° C. for 10 minutes in 500 mL glass beakers, and the amount of curry stain removed was assessed using the Konica Minolta spectrophotometer. A second control was conducted in the same wash conditions, using 1 mL pH=10 with 10 g/L sodium perborate tetrahydrate and no beads. A third control was similarly conducted at pH=10 (softened water), using neither beads nor sodium perborate tetrahydrate. The results are shown in Table 15.

TABLE 15

| | ΔL* |
|---|---|
| Soft water only | 4.1 |
| Sodium perborate tetrahydrate, no beads | 4.6 |
| Unhydrolysed Nylon 6 beads | 4.8 |
| Hydrolysed Nylon 6 beads | 7.0 |

### Example 13

### Layered Beads

This Example is a comparative example and corresponds to Example 4 of WO-20141006424-A. The data in the table below demonstrate the performance difference of the present invention over the layered beads of this prior art.

### 1) Bead Preparation

15 g of pure Nylon beads were soaked sequentially in 1 mg/mL PEI (polyethyleneimine), then 1 mg/mL Stainzyme Plus 12L® (an amylase commercially available from Novozyme), then 1 mg/mL PEI, then 1 mg/mL Stainzyme Plus 12L®. Each soak lasted for 2 hours, at 21° C. with gentle agitation. In each case, 11.25 ml of solution was used, sufficient to cover the beads in the vial. Softened water was used for dilution of PEI and Stainzyme. The beads were rinsed in soft water and dried with a paper towel between sequential washes. Fresh PEI and Stainzyme solutions were used for the first and third, and second and fourth soaks respectively. The beads were rinsed in soft water and dried with a paper towel prior to washes. The beads were then finally rinsed in soft water and dried.

### 2) Enzyme Adsorption and Application for Soil Removal

5 cm×5 cm starch-stained swatches (10R, WFK) were washed in 100 ml soft water using 15 g of layered beads at 21° C. for 10 minutes in 500 mL glass beakers, and the amount of starch stain removed was assessed using the Konica Minolta spectrophotometer. A control test with soft water only, and a control test with just the Stainzyme enzyme only no beads, were also undertaken. In order to determine the quantity of Stainzyme solution to be used in the enzyme-only control, the beads were weighed between consecutive soaks during their preparation, and the mass of 1 mg/mL Stainzyme solution adsorbed calculated as 0.26 g. The method of adsorption and desorption of Stainzyme enzyme for hydrolysed and unhydrolysed beads is included in Example 6 herein. The results are shown in Table 16.

TABLE 16

| | ΔL* | | |
|---|---|---|---|
| Starch stain swatch | Wash 1 | Wash 2 | Wash 3 |
| Soft water only | 0.0 | 0.0 | 0.0 |
| No beads, Stainzyme enzyme only | 0.4 | 0.6 | 0.7 |
| Layered nylon 6 beads | 2.9 | 1.7 | 0.7 |
| Unhydrolysed beads with Stainzyme | 7.9 | 8.4 | 11 |
| Hydrolysed beads with Stainzyme | 11.8 | 12.4 | 12.3 |

The results demonstrate that the hydrolysed beads and the unhydrolysed beads within the scope of the invention are superior in removing starch comparing to the layered nylon beads of the prior art.

### Example 14

### Cellulase Adsorption

12 mL of a solution of 1 g of cellulase ((Cellosoft 19500; Novozymes) in 100 mL of pH 4/4.5 soft water solution (citric acid, Fluka Analytical, UK) was adsorbed onto 15 grams of beads (both hydrolysed and unhydrolysed bead samples were prepared). The Bio-Rad Assay was performed to determine the quantity of enzyme adsorbed onto the hydrolysed and unhydrolysed beads. The data in table 17 indicate that cellulase is adsorbed onto both types of bead but preferentially onto the hydrolysed bead surface (70.3% of the enzyme is adsorbed from solution).

TABLE 17

| | % adsorption of cellulase onto the bead surface |
|---|---|
| Hydrolysed nylon 6 beads | 70.3 |
| Unhydrolysed nylon 6 beads | 33.3 |

1. A method for applying a treatment agent to a substrate using solid polymeric treatment particles, wherein the substrate is a textile or a fibre, said method comprising:
a) providing solid polymeric treatment particles obtainable by at least partially coating solid polymeric particles with the treatment agent in the presence of an aqueous liquid medium, the liquid medium having a first pH, wherein:
(i) the surface of the solid polymeric particles has a net positive or net negative charge at the first pH; and
(ii) the treatment agent has a net positive or net negative charge within its chemical structure at the first pH;
wherein the sign of the net charge on the surface of the solid polymeric particles at the first pH is opposite to the sign of the net charge of the treatment agent at the first pH; and
b) contacting the substrate with the solid polymeric treatment particles from step a), in an aqueous liquid medium under conditions such that the treatment agent is released from the solid polymeric treatment particles;
wherein the treatment agent is released from the solid polymeric treatment particles by contacting the solid polymeric treatment particles with the substrate in an aqueous liquid medium at a second pH at which the net charge on the surface of the solid polymeric particles or the net charge on the treatment agent has changed such that the signs of the net charge on the surface of the solid polymeric particles is the same as the sign of the net charge of the treatment agent or such that there is no net charge on the surface of the solid polymeric treatment particles or no net charge of the treatment agent; or
wherein the treatment agent is released from the solid polymeric treatment particles by contacting the solid polymeric treatment particles with the substrate in an aqueous salt solution.

2. A method for applying a treatment agent to a substrate, wherein the substrate is a textile or a fibre, said method comprising providing solid polymeric treatment particles comprising solid polymeric particles and a treatment agent, wherein the treatment agent is ionically bound to the surface of the solid polymeric particles, and contacting said solid polymeric treatment particles with the substrate in an aqueous liquid medium under conditions such that the treatment agent is released from the solid polymeric treatment particles;
wherein the treatment agent is released from the solid polymeric treatment particles by changing the pH of the aqueous liquid medium; or
wherein the treatment agent is released from the solid polymeric treatment particles by contacting the solid polymeric treatment particles with the substrate in an aqueous salt solution.

3. A method according to claim 2, wherein the solid polymeric treatment particles are obtainable by at least partially coating solid polymeric particles with the treatment agent in the presence of an aqueous liquid medium, the aqueous liquid medium having a first pH, wherein:
(i) the surface of the solid polymeric particles has a net positive or net negative charge at the first pH; and
(ii) the treatment agent has a net positive or net negative charge within its chemical structure at the first pH;
wherein the sign of the net charge on the surface of the solid polymeric particles at the first pH is opposite to the sign of the net charge of the treatment agent at the first pH.

4. A method according to claim 1, wherein the solid polymeric treatment particles are contacted with the substrate with agitation.

5. A method according to claim 1, for laundry and textile washing, treatments, pre-treatments and post-treatments of textiles and fibres, and dishwashing.

6. A method according to claim 1, wherein said liquid medium has a first pH and the treatment agent comprises at least one ionic group at the first pH.

7. A method according to claim 1, wherein the treatment agent is selected from a surfactant, a buffer, a sequestrant, a builder, a dye, a singlet oxygen generator, a bleach compound, a bleach activator, a bleach catalyst, a dispersant, an optical brightener, an antioxidant, an enzyme, a fragrance, a cyclodextrin, an antistatic agent, a UV protector, an antimicrobial agent, a fabric conditioner, an insecticide, an insect-repellant, a flame retardant, a water-repellant, an oxide or a mixture thereof.

8. A method according to claim 1, wherein the solid polymeric particles have (i) an average mass of from about 1 mg to about 1000 mg; and/or (ii) an average volume in the range of from about 5 to about 500 mm³; and/or (iii) an average surface area of from 10 mm² to 500 mm² per particle; and/or (iv) an average particle size of from 1 mm to 20 mm, more preferably from 1 mm to 10 mm.

9. A method according to claim 1, wherein the solid polymeric particles comprise or consist of a polyalkene, a polyamide, a polyester or a polyurethane,

10. A method according to claim 9, wherein the solid polymeric particles comprise or consist of a polyamide.

11. A method according to claim 10, wherein the solid polymeric particles comprise or consist of a polyamide selected from nylon 6 or nylon 6,6.

12. A method according to claim 10, wherein the solid polymeric particles are activated with an acid, a base or an oxidising agent or a combination thereof.

13. A method according to claim 12, wherein the wherein the solid polymeric particles are activated with hydrochloric acid having a molar strength of from 2.0 to 5 M.

14. A method according to claim 1, wherein the solid polymeric particles have an isoelectric point in the range of from pH 3 to pH 7, more preferably in the range of from pH 4 to pH 6, more preferably from pH 5 to pH 6.

15. A method according to claim 1 any one of the preceding claims, wherein the pH of said liquid medium having a first pH is at least 1 pH unit above or 1 pH unit below the isoelectric point of the solid polymeric particles and more preferably at least 2 pH units above or 2 pH units below the isoelectric point of the solid polymeric particles.

16. A method according to claim 1, wherein the isoelectric point of the solid polymeric particles is from 4 to 6 or from 4.5 to 6.5; and the pH of said liquid medium having a first pH is from 7 to 14 or from 1 to 5; provided that the isoelectric point is not the same as the first pH.

17. A method according to claim 1, wherein the treatment agent is released from the solid polymeric treatment particles by contacting the solid polymeric treatment particles with the substrate in an aqueous liquid medium at a second pH at which the net charge on the surface of the solid polymeric particles or the net charge on the treatment agent has changed such that the signs of the net charge on the surface of the solid polymeric particles is the same as the sign of the net charge of the treatment agent or such that there is no net charge on the surface of the solid polymeric treatment particles or no net charge of the treatment agent.

18. A method according to claim 17, wherein the first and second pH are selected such that:
A) the first pH is from 7 to 14; more preferably from 8 to 12; and the second pH is from 1 to 5, more preferably from 2 to 5; or
B) the first pH is from 1 to 5, more preferably from 2 to 5 and the second pH is from 7 to 14; more preferably from 8 to 12.

19. A method according to claim 1, wherein the treatment agent is released from the solid polymeric treatment particles by contacting the solid polymeric treatment particles with the substrate in an aqueous salt solution.

20. A method according to claim 1, wherein the solid polymeric treatment particles are contacted with the substrate at a temperature of from 20° C. to 90° C. and more preferably at a temperature of from 20° C. to 60° C.

21. A method according to claim 1, wherein the solid polymeric treatment particles are contacted with the substrate for a duration of from 1 minute to 3 hours and more preferably for a duration of from 5 minutes to 1.5 hours.

22. A method according to claim 1, wherein the solid polymeric treatment particles are contacted with the substrate in the presence of a surfactant.

23. A method according to claim 1, wherein the solid polymeric treatment particles are contacted with the substrate in a more dilute concentration than is used for the preparation of the solid polymeric treatment particles.

24. A method according to claim 1 further comprising the following steps c), d) and e):
c) separating the solid polymeric particles from the substrate and the aqueous liquid medium;
d) optionally cleaning the solid polymeric particles to remove any residual treatment agent;
e) re-using the solid polymeric particles in a method for applying a treatment agent to a fresh substrate as defined above.

25. A method according to claim 1, wherein the solid polymeric treatment particles may be contacted with the substrate using an apparatus which comprises:
1. a housing;
2. a rotatable drum;
3. a motor configured so as to be capable of rotating the rotatable drum;
4. a pump for transporting the solid polymeric treatment particles into said drum; and
5. a sump for collecting the solid polymeric treatment particles once the treatment is complete.

26. (canceled)

27. A method according to claim 1, wherein unused treatment agent which has not been successfully been applied to the substrate is recovered.

28. (canceled)

29. A method according to claim 1, wherein the treatment agent is released from the solid polymeric treatment particles by contacting the solid polymeric treatment particles with the substrate in an aqueous liquid medium at a second pH at which the net charge on the surface of the solid polymeric particles or the net charge on the treatment agent has changed such that the signs of the of the net charge on the surface of the solid polymeric particles is the same as the sign of the net charge of the treatment agent or such that there is no net charge on the surface of the solid polymeric treatment particles or no net charge of the treatment agent, and wherein:
i. the solid polymeric particles comprise or consist of a polyamide;
ii. the solid polymeric particles have an isoelectric point at a pH of from 4 to 6;
iii. the solid polymeric particles carry both cation-forming and anion-forming groups;
iv. the substrate comprises or consists of a fibre (more preferably the substrate comprises or consists of a textile);
v. the first and second pH are selected such that:
A) the first pH is from 7 to 14; more preferably from 8 to 12; and the second pH is from 1 to 5, more preferably from 2 to 5; or
B) the first pH is from 1 to 5, more preferably from 2 to 5 and the second pH is from 7 to 14; more preferably from 8 to 12.
vi. the treatment agent has one or more ionic groups.

30-34. (canceled)
